(19) United States
(12) Patent Application Publication
(10) Pub. No.: US 2008/0075767 A1
(43) Pub. Date: Mar. 27, 2008

(54) IBUPROFEN-CONTAINING LIQUID FILLED HARD CAPSULES

(76) Inventors: Xiao Pin Jin, Mississauga (CA); Chandir Ramani, Brampton (CA); Lester David Fulper, Loveland, OH (US); Tak Kau Uri Law, Thornhill (CA); Dawn Denise Downey, West Chester, OH (US); Anil Kane, Mississauga (CA); David W. Wynn, Huntingdon Valley, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/768,229

(22) Filed: Jun. 26, 2007

Related U.S. Application Data
(60) Provisional application No. 60/806,315, filed on Jun. 30, 2006.

(51) Int. Cl. A61K 9/66; A61K 31/192; A61P 25/04

(52) U.S. Cl. 424/455; 514/570

(57) ABSTRACT

The present invention relates to ibuprofen-containing pharmaceutically acceptable solutions for filling hard capsules.

### FIELD OF THE INVENTION

The present invention relates to pharmaceutically acceptable solutions for filling hard capsules, hard capsules containing these solutions, and a process for preparing these hard capsules.

### BACKGROUND OF THE INVENTION

Ibuprofen (2-(4-isobutylphenyl)propionic acid is a drug which has anti-inflammatory and analgesic properties. It is used for the treatment of rheumatoid arthritis or other inflammatory diseases of joints, soft tissue rheumatism and gout.

Ibuprofen, although it is soluble in some physiologically compatible solvents, will immediately precipitate upon the addition of small amounts of water or when the solution is introduced into an aqueous medium at a low pH such as, for example, an artificial gastric juice. When such a solution, upon oral administration, gets into the stomach, the ibuprofen precipitates so that it will be barred from a quick resorption.

During most of the 20th century, hard gelatin capsules were a popular dosage form for prescription and over-the-counter (OTC) drugs. Capsules are hard shell compartments made of two halves, including a body and a cap, wherein the cap partially and snugly overlaps with the body to enclose a dosable drug ingredient therein. The enclosed dosable ingredient is most often is a powder, liquid, paste or similar nonsolid form. Capsules have the additional advantage of allowing for the powder to be in an uncompressed form, since certain active ingredients cannot be easily compressed into a tablet form, and dissolve readily in gastric fluids.

Generally, empty hard shell capsules are produced by a conventional dip-molding process such as that which is described on page 182 of “Pharmaceutical Dosage Forms and Drug Delivery Systems, 7th Ed.”, (1999) by Howard C. Ansel, Loyd V. Allen Jr., and Nicholas G. Popovich, published by Lippincott Williams & Wilkins, Baltimore, Md. Consumers have found that such capsules are aesthetically pleasing, easy to swallow and mask the medicine taste of the drug contained therein. In addition, the bodies and caps of such capsules are often produced in different colors, resulting in a bi-colored capsule product having enhanced aesthetic appeal, as well as improved product identification and brand recognition by consumers. Many patients preferred capsules over coated or uncoated tablets, prompting pharmaceutical manufacturers to market certain products in capsule form even when they were also available in tablet form.

Several materials are known to be used to form the shell. Gelatin has been adopted as the main material of these capsules due to its excellent characteristic as a gelatinizer. The gelatin dissolves under high concentration into water of a high temperature and quickly gels at room temperature conditions of about 25° C. The thickness of the film made by the gelatin becomes uniform. However, gelatin is one of the proteins derived from animals; most commonly from the bones of bovine animals; and can be viewed as unstable from a chemical viewpoint due to its tendency to cross-link, which can slow the dissolution rate, can be microbally unstable, and has a risk of TSE. As a result, several materials have been examined as a substitute for the gelatin in two-piece hard capsules. Hydroxypropylmethyl cellulose (HPMC) or hypromellose is used as an alternative material for two-piece capsule. HPMC capsules have been developed for both pharmaceutical products and dietary supplements.

Liquid filled capsules can be manufactured in hard and soft forms, and allow for active ingredients to be solubilized or suspended in a liquid medium within the capsule fill. Liquid filled capsules are viewed by consumers in many instances as superior in dissolution characteristics to powder filled capsules. When active ingredients are pre-solubilized in the fill of a liquid filled capsule, the active may not require further dissolution in a gastric liquid medium, or may allow for faster emptying of the active from the stomach to the duodenum and small intestine where the drug is absorbed. Therefore, certain active ingredients allow for faster bioavailability when in liquid filled capsule form.

Liquid filled hard capsules (LFHC) are currently being used with low-solubility molecules, high-potency molecules, molecules susceptible to oxidation, molecules exhibiting low melting points, and molecules requiring controlled/sustained release formulations. One additional advantage of liquid filled hard capsules over liquid filled soft capsules is that there is an stronger barrier against tamperability.

It is one object of the present invention to provide a pharmaceutically acceptable clear solution for filling hard, preferably transparent gelatin capsules which overcomes the above disadvantages of the prior art. The filled capsules should be usable as medicaments that can be readily taken and that may contain high concentrations of ibuprofen in a carrier, that are simple to prepare and that will quickly display a high activity. The solutions for filling hard gelatin capsules should show an increased stability and bioavailability of ibuprofen.

### SUMMARY OF THE INVENTION

A pharmaceutically acceptable solution for filling a hard capsule comprising, consisting of, and/or consisting essentially of, based upon the total weight of the solution:

(a) from about 45 to about 75% by weight ibuprofen,

(b) from about 3 to about 5% by weight of an alkylizing agent, and

(c) from about 30 to about 46% by weight of a solvent selected from the group consisting of a vegetable oil, a polyglycolized glyceride, a combination of polyethylene glycol and a polyoxyethylene stearate, and combinations thereof,

wherein the molar ratio between the alkylizing agent and ibuprofen is about 1:about 1.

A pharmaceutically acceptable solution for filling hard capsules comprising, consisting of, and/or consisting essentially of, based upon the total weight of the solution:

(a) from about 60% by weight ibuprofen,

(b) from about 3% by weight of potassium hydroxide and

(c) from about 37% by weight of a combination of polyethylene glycol and a polyoxyethylene stearate,

wherein the molar ratio between the potassium hydroxide and ibuprofen is about 1:about 1.

### DETAILED DESCRIPTION OF THE INVENTION

As used herein, “liquid-filled” means that the overall physical form of the filling is a liquid at room temperature. The expression “liquid-filled” is intended to include solutions, suspensions or mixtures of liquids and solids which have the overall characteristics of a liquid.

The present invention relates to a liquid filled pharmaceutical capsule dosage form that includes a pharmaceutically effective amount of ibuprofen and a non-aqueous liquid carrier. The present invention also relates to a liquid filled pharmaceutical capsule dosage form that includes a pharmaceutically effective amount of acetaminophen and a non-aqueous liquid carrier.

In particular, the present invention relates to a pharmaceutical hard capsule dosage form that is stable under accelerated stability conditions.

Examples of active ingredients useful in the present invention include propionic acid derivatives, which are a well known class of analgesic compounds. As used herein propionic acid derivatives are understood to include, but are not limited to, ibuprofen, naproxen, benoxaprofen, naproxen sodium, flurbiprofen, fenoprofen, fenbuprofen, ketoprofen, indoprofen, pirprofen, carpofen, oxaprofen, pranoprofen, microprofen, tioxaprofen, suproprofen, alminoprofen, tiaprofenic acid, fluprofen and bucloxic acid. The structural formula is set forth in U.S. Pat. No. 4,923,898, hereby incorporated by reference. Propionic acid derivatives as defined herein are defined as pharmaceutically acceptable analgesics/non-steroidal anti-inflammatory drugs having a free —CH(CH3)COOH or —CH2CH2COOH or a pharmaceutically acceptable salt group, such as —CH(CH3)COO—Na+ or CH.sub.2CH2COO—Na+, which are typically attached directly or via a carbonyl functionality to an aromatic ring system.

Propionic acid derivatives are typically administered on a daily basis, with the daily dose ranging from about 25 to about 2000 milligrams, preferably from about 100 to about 1600 milligrams and most preferably from about 100 to about 1200 milligrams. Ibuprofen is typically administered on a daily basis, with a daily dose ranging from about 50 to about 2000 milligrams, preferably from about 100 to about 1600 milligrams and most preferably from about 200 to about 1200 milligrams. In one embodiment of this invention each individual hard shell liquid filled capsule contains about 50 to about 400 milligrams or about 100 milligrams to about 200 milligrams of ibuprofen.

Ibuprofen is a widely used, well known non-steroidal anti-inflammatory propionic acid derivative. Ibuprofen is chemically known as 2-(4-isobutylphenyl)-propionic acid. As used herein ibuprofen is understood to include 2-(4-isobutylphenyl)propionic acid as well as the pharmaceutically acceptable salts. Suitable ibuprofen salts include arginine, lysine, histidine, as well as other salts described in U.S. Pat. Nos. 4,279,926, 4,873,231, 5,424,075 and 5,510,385, the contents of which are incorporated by reference.

The amount of ibuprofen used in the present invention is a pharmaceutically effective amount. This amount can be from about 45 to about 75% by weight, preferably about 50 to about 65% by weight, most preferably about 60% by weight, with respect to the total weight of the hard capsule liquid fill solution.

Other active agents that may be useful in the present invention include pseudoephedrine, phenylephrine, phenylpropanolamine, chlorpheniramine maleate, clofedianol, dextromethorphan, diphenhydramine, famotidine, loperamide, ranitidine, cimetidine, astemizole, terfenadine, fexofenadine, cetirizine, mixtures thereof and pharmaceutically acceptable salts thereof.

Examples of non-aqueous carriers or vehicles, e.g., solvents, include
the chemical class of vegetable oils, vegetable oil triglycerides and triacylglycerols, specifically, for example, corn oil;
the chemical class of polyglycolized glycerides, specifically, for example, lauryl macrogol 32-glycerides and steroyl macrogol 32-glycerides, such as those sold under the tradename Gelucire® 44/14 and Gelucire® 50/13 available from the Gattefosse Corporation; in addition, the chemical class of glycerol esters of fatty acids such as those sold under the tradename Gelucire® 33/01, Gelucire® 39/01, and Gelucire® 43/01 available from the Gattefosse Corporation, and mixtures thereof;
the chemical class of neutral oils and triglycerides, specifically, for example, medium chain triglycerides, fractionated coconut oil, caprylic and capric triglycerides such as those sold under the tradename Miglyol® 812 available from the Condea Vista Corporation, and mixtures thereof;
the chemical class of polyethylene glycol and polyoxyethylene stearates, specifically, for example, polyethylene glycol 15 hydroxystearate as sold under the tradename Solutol® HS 15 available from the BASF Corporation, and mixtures thereof;
the chemical class of purified vegetable, soybean and egg yolk lecithin, specifically, for example, phosphatidyl choline and 1,2-diacyl-sn-glycero-3-phosphoryl choline such as those sold under the tradename Phospholipon® 90 G available from the American Lecithin Company, and mixtures thereof;
the chemical class of lecithin combined in propylene glycol, specifically, for example, standardized mixtures of phosphatidylcholine, propylene glycol, mono- and di-glycerides, ethanol, soya fatty acids and ascorbyl palmitate, such as those sold under the tradename of Phosal® 50 PG available from the American Lechitin Corporation;
the chemical class of capryl-caproyl macrogol-8-glyceride and caprylo caproyl macrogol-8 glycerides such as those sold under the tradename Labrasol® available from the Gattefosse Corporation, and mixtures thereof;
the chemical class of polyethoxylated hydrogenated castor oil, specifically, for example, glycerol-polyethylene glycol oxystearate, such as those sold under the tradename Cremophor® RH 40 and Cremophor® EL available from the BASF Corporation, and mixtures thereof;
alkalizing agents including potassium hydroxide, sodium hydroxide, magnesium hydroxide, calcium hydroxide, potassium acetate, sodium acetate, magnesium acetate, calcium carbonate, calcium oxide, calcium phosphates, magnesium carbonate, magnesium oxide, magnesium phosphates, magnesium hydroxide carbonate, magnesium aluminum silicate, magaldrate, bentonite, zeolites, magnesium silicates, hydrotalcite, dihydroxyaluminum sodium carbonate, ammonium hydroxide, ammonium bicarbonate, ammonium carbonate, ethanolamine, diethanolamine, triethanolamine, sodium bicarbonate, potassium bicarbonate, magnesium hydroxide, aluminum hydroxide, magnesium phosphates, tetrasodium ethylenediaminetetraacetic acid and its hydrates and mixtures thereof, and
mixtures or combinations of any of the above.

The alkalizing agent used in the present invention is an amount of from about 3 to about 7% by weight, preferably about 4% to about 6% by weight, more preferably about 5.5% by weight with respect to the total weight of the solution.

The most preferred alkali hydroxide according to the invention is potassium hydroxide (KOH).

In a preferred embodiment of the invention the alkalizing agent is used in an amount of from about 0.8 moles to about 1.2 moles per about 1 mole ibuprofen, most preferably about 1 mole alkalizing agent to about 1 mole ibuprofen.

In one aspect the present invention provides an ibuprofen-containing hard gelatin capsule containing the aforementioned solution comprising a pharmaceutically effective amount of ibuprofen, an effective amount of an alkalizing agent and, optionally, water and/or other ingredients.

If water is added to solution, it is added in an amount that is in an amount that is less than about 4% by weight, from about 1.5 to about 3% by weight, with respect to the total weight of the solution. In one embodiment the capsule fill solution is substantially free of water, which as used herein, is defined as less than about 4% by weight of water.

The hard capsule is a system comprised of the ibuprofen-containing solution formulation, the shell used to encapsulate the ibuprofen-containing solution and an optional band that seals the seam around the hard capsule. As such, not only is the filled ibuprofen formulation critical to produce the desired bioavailability characteristics but the gelatin formulation and the dealing band formulation are also critical as it must be compatible with the ibuprofen formulation. The potential fill-shell interactions could result in both physical and chemical capsule instability. Accordingly, the formulation utilized to form the capsule for the ibuprofen dosage form is also important to the present invention.

Therefore, the present invention utilizes a hard capsule that provides physical and chemical stability to the ibuprofen-containing solution of the present invention.

The capsule formulations can also include other suitable additives such as preservatives and/or coloring agents which are utilized to stabilize the capsule and/or impart a specific characteristic such as color or look to the capsule. The capsule may also contain flavorants, sensates, fragrances, acidulants such as citric, fumaric or malic acid; cooling agents such as menthol or non-volatile coolers such as but not limited to Cooler #2, commercially available from International Flavors and Fragrances; and sweeteners such as but not limited to sucralose, aspartame, saccharine, acesulfame potassium and related salts and derivatives thereof.

In one particular embodiment a hard gelatin capsule can be differentiated from a soft gelatin capsule by determining the elongation at break value of the capsule material. In one embodiment, whereas a soft gelatin liquid filled capsule material possesses an elongation at break value of at least about 50%, a hard gelatin liquid filled capsule material possesses an elongation at break value of between about 1% and about 40%, when film samples of each layer are independently tested in accordance with that described in the American Society for Testing Materials (ASTM) D882 test measurement. According to this test method, a film sample is cast and cut or stamped using an ASTM D1708 Stamp mold, then inserted into a press such as the Punch Press Model B No. 8463 as produced by the Naef Corporation. The film sample is then placed between two grippers on a texture analyzer, such as the model TA-XT2i (HR) available from Texture Technologies Corporation, which elongates the film from two ends and determines the percentage value at break.

In another embodiment a soft gelatin liquid filled capsule can be deformed upon compression of at least of at least 2% of the diameter of the shortest axis of the capsule without rupture, whereas a hard gelatin liquid filled capsule cannot be deformed of greater than about 0.5% percent of the diameter of the shortest axis without rupture.

The liquid fill hard capsules may be made by any method known in the art. For example, a Liqfil Super 40 that fills powders, pellets, beads, pastes, oils, and liquids at speeds of 40,000 capsules per hour, and incorporates a hot-air purge system to prevent leakage and bubbles, along with an add-on cooling tower to protect capsules can be used. Machines that combine filling and sealing operations are a recent developments for capsules. Lab-scale machines that fills and seals liquids into two-piece capsules at speeds of up to 3000 capsules per hour are available. A Capsugel's (Greenwood, S.C.) Liquid Encapsulation Microspray Sealing (LEMS) production-scale machine seals up to 30,000 capsules per hour.

Various methods can be used to seal the hard gelatin capsules according to the invention.

The banding of hard gelatin capsules is well-known in the art. The capsules are first rectified and then passed once or twice over a wheel that revolves in a gelatin bath. An amount of gelatin is picked up by the serrated wheel and applied to the junction of the cap and body. The capsules remain in individual carries for drying. The sealing band may be made up of gelatin or other water soluble film forming polymers such as but not limited to hypromellose; hydroxypropylcellulose; polyvinyl pyrrolidone, gellan gum, microcrystalline cellulose, carageenan; polyvinyl alcohol, polyethylene glycol and related co-polymers.

According to the invention the hard gelatin capsule sealing is preferred which is based upon the lowering of the melting point of gelatin by the application of moisture to the area between the capsule body and cap.

In a preferred embodiment of the present invention a method is thus contemplated where the capsules are filled and then sealed by spraying a small amount of a water/ethanol mixture at the cap and body interface followed by warming to fuse the two capsule part together.

Instrumentation for performing the encapsulation according to the above methods is commercially available.

Using the solution of the present invention, it is possible to prepare a unit dose of ibuprofen in a two piece hard capsule, wherein the fill solution contains a therapeutically effective amount of ibuprofen dissolved within. The dosages administered will vary depending upon the acidic pharmaceutical agent employed, the mode of administration the treatment desired, the size, age, and weight of the patient being treated and the like.

### EXAMPLES

The invention will now be illustrated by, but is not intended to be limited to, the following examples. In these examples it is understood that unless noted otherwise, all parts, percentages and ratios are by weight.

### Example 1

### Ibuprofen Vehicle Screening

Initially, ibuprofen United States Pharmacopeia grade (USP) powder was mixed at room temperature with individual excipients being considered as shown in Table 1. Ibuprofen was incrementally added and mixed until a precipitate was observed. At that point, the suspension was placed on a hot plate to melt in ibuprofen and mix to form a clear solution. Observations were made daily to determine if the mixture would remain a solution or precipitate out within an approximately 24 hour period. If the ibuprofen remained in solution at the subsequent observation period, more active pharmaceutical ingredient (API) (in this example, ibuprofen) was added and the resulting suspension was re-heated to form a solution. Table 1 shows the maximum percentage of API where it was demonstrated that API remained in solution. The third column shows that percentage of API added where a precipitate formed within approximately 24 hours after the mixture was cooled to room temperature.

TABLE 1

| | Approx. Max. | Approx. Percentage |
|---|---|---|
| | Percentage of API | of API that Resulted |
| Excipient | in Solution Tested | in Precipitate |
| Solutol HS 15 | 40 | 50 |
| Hexylene Glycol | 31 | 50 |
| Cremophor RH40 | 29 | 35 |
| Polyoxymer 124 | 27 | 30 |
| PEG 400 | 23 | 33 * |
| Labrasol | 22 | 33 |
| Capmul PG8 | 22 | 32 |
| Macol LA 4 | 22 | 32 |
| Tween 80 | 21.5 | 27 * |
| Tween 20 | 21 | 26 * |
| Capryol 90 | 21 | 32 |
| Propylene Glycol | 17 | 28 * |
| Schercemol DIA | 18 | 30 |
| Captex 200P | 15 | 27 |
| Labrafac PG | 15 | 27 |
| Labrafil M 1944 CS | 15 | 27 |
| Labrafil M 2125 CS | 15 | 27 |
| Labrafac Hydro WL 1219 | 13 | 32 |
| Miglyol 812 | 13 | 27 |
| Myglyol 840 | 13 | 27 |
| MYVACET 9-45V | 13 | 23 |
| Akomed E | 13 | 20 |
| Softigen 701 | 9 | 27 * |
| Plurol Oleique CC 497 | 9 | 27 |
| Schercemol TN | 9 | 23 |
| Corn Oil | 7 | 22 |
| Olive Oil | 7 | 22 |
| Akomed R | 7 | 13 |
| Captex 355 | 7 | 15 |
| Labrafac CC | 7 | 15 |
| Neobee M-5 | 7 | 13 |
| Imwitor 988 | 3 | 27 |
| Surfactol 365 | 2.5 | 27 * |
| Softisans 645 | 2.5 | 26 |
| SPAN 80 | 2 | 25 |
| Castor Oil | Initial test | 3 |
| | precipitated | |
| Imwitor 491 | Initial test | 2.5 |
| | precipitated | |
| Glycerin | Initial test | 2.5 |
| | precipitated | |

* This appears to be close to the saturation point. Only a few crystals observed at this concentration of ibuprofen.

### Example 2

### Ibuprofen Lysinate Vehicle Screening

Ibuprofen lysinate was screened for solubility in various vehicles. Limited solubility was observed; however, these studies were conducted mainly in anhydrous systems. Increased solubility would be expected in binary systems that include water and would allow adjustment of pH. See summary in Table 2.

TABLE 2

| | Approximate Percentage of | |
|---|---|---|
| | IBL salt added in powder | |
| Excipient | form to excipient and heated | Comments |
| PEG 400 | 2.4 | Suspension |
| Glycerin | 2.2 | Suspension initially, |
| | | dissolves after several days. |
| Propylene glycol | 2.4 | One large piece of IBL that won't |
| | | dissolve, the rest is still clear |
| | | solution after approx. 1 week |
| Cremophor RH 40 | 2.1 | Won't dissolve. Stays in suspension. |
| Polyoxamer 124 | 2.4 | Won't dissolve. Stays in suspension. |
| Tween 80 | 2.4 | Won't dissolve. Stays in suspension. |
| Tween 20 | 2.4 | Won't dissolve. Stays in suspension. |
| Hexylene Glycol | 2.4 | Won't dissolve. Stays in suspension. |
| Labrasol | 4.6 | Won't dissolve. Stays in suspension. |
| Phosal 50 PG | 2.5 | Color becomes darker and pieces of |
| | | undissolved material remain at the |
| | | bottom of the vessel. |
| PEG 400 saturated | 3.2 | Added in a total of 2 grams water |
| with Ibuprofen, 33% | | with IBL to get it to dissolve. |
| | | Precipitate started to form after |
| | | approximately 72 hours. |
| Propylene Glycol | 3.4 | Dissolved into solution. Added |
| saturated with | | another 3.3% after approx. 24 hours |
| Ibuprofen, 28% | | and observed a few crystals |
| | | precipitating the following day. |
| Tween 80 saturated | 3.5 | Won't dissolve. Stays in suspension. |
| with Ibuprofen, 27% | | |
| Tween 20 saturated | 3.4 | Won't dissolve. Stays in suspension. |
| with Ibuprofen, 26% | | |
| Softigen 701 saturated | 3.5 | Won't dissolve. Stays in suspension. |
| with Ibuprofen, 27% | | |
| Surfactol 365 saturated | 3.5 | Won't dissolve. Stays in suspension. |
| with Ibuprofen, 27% | | |

### Example 3

### Ibuprofen in Phosal/Solubilizer Combination Vehicle

Table 3 shows mixtures that have been examined for ibuprofen in combination with lecithin/phosphatidylcholine based systems. Systems based on phosphatidylcholine (Phosal) are structured systems that form micelles. The scale that was investigated did not allow for evaluation of the affect of mixing. Since mixing can influence the formation of the micelle structure, further opportunities may exist to improve the performance of these systems through mixing studies.

TABLE 3

| | Approx. % API in | |
|---|---|---|
| Excipient | Solution - Resulted in | |
| Combination | Precipitated | Comments |
| 50% Phosal 50 PG/ | 39 | 31% ibuprofen in this combination did not |
| 50% Propylene Glycol | | show a precipitate after approx. 72 hours. |
| 50% Phosal 53 MCT/ | 39 | 31% ibuprofen in this combination did not |
| 50% Propylene Glycol | | show a precipitate after approx. 72 hours. |
| 50% PEG 400/50% | 33 | Very few crystals observed in precipitate |
| Phosal 53 MCT | | after 24 hours |
| 50% PEG 400/50% | 33 | Very few crystals observed in precipitate |
| Phosal 50 PG | | after 24 hours |
| 50% Capmul PG8/ | 33 | Some precipitate after 24 hours. |
| 50% Phosal 50 PG | | |
| 50% Capmul PG8/ | 33 | Precipitated after 24 hours. |
| 50% Phosal 53 MCT | | |
| 20% Tween 20/40% Phosal | 40 | Precipitated out within 24 hours. |
| 53 MCT/40% Ibuprofen | | |
| 50% Phosal 50 PG/ | 40 | Excipient mix is clear yellow. ibuprofen |
| 50% Labrasol | | |
| 50% Phosal 53 MCT/ | 40 | Also forms a crystal type precipitate within |
| 50% Labrasol | | one |
| 50% Phosal 53 MCT/ | N/A | Excipient combination solidified within 72 |
| 50% Cremophor RH 40 | | hours. |
| 50% Phosal 50 PG/ | N/A | Phase separation observed. |
| 50% Soybean Oil | | |

### Example 4

### Ibuprofen Combination Vehicle Screening

The combination vehicle screening for ibuprofen was initiated with the following objective:
Repeat two formulations—EP134452 and WO2069936;
Evaluate ibuprofen solubility in Corn oil (lipophilic) and PEG 400 (hydrophilic) combining with alkalizing agents, KOH and KHCO₃; and
Enhance the solubility with polyvinylpyrrolidone. The Labrasol/KHCO₃, Corn oil/KOH and PEG/KOH vehicle systems provided promising results. Up to 40% ibuprofen was dissolved.
   Sample Preparation Procedure:
   1) Add ibuprofen into solvent vehicle.
   2) Add KOH or KHCO₃ as solids to the mixture of Step 1.
   3) Heat the mixture until it is a clear solution.
   4) Keep samples at room temperature for approximately 24 hours.
   5) Visually evaluate the solubility of the samples.
   6) Add additional ibuprofen to the samples that are still in clear solution.
   7) Repeat step 3 to 5.

See Table 4 for a summary of the results.

TABLE 4

| | 1 | 2 | | | | |
|---|---|---|---|---|---|---|
| | (EP1344523) | (WO02069936) | 3 | 4 | 5 | 3A |
| Ingredient | % (w/w) | % (w/w) | % (w/w) | % (w/w) | % (w/w) | % (w/w) |
| Ibuprofen | 47.62 | 37.34% | 35% | 35% | 35% | 35% |
| | (200 mg) | (200 mg) | | | | |
| PEG400 | | | | 65% | 45% | |
| Corn oil | | | 65% | | | 60% |
| KOH | 3.88% | | 0.3 mol | 0.3 mol | | |
| | (16.29 mg) | | | | | |
| KHCO3 | | 5.43% | | | 5.43% | 5.43% |
| | | (29.1 mg) | | | | |
| PVP 29/31 | | | | | 20% | |
| Cremophor | 33.5% | | | | | |
| RH40 | (140.71 mg) | | | | | |
| Miglyol | 15% (63 mg) | | | | | |
| 812 | | | | | | |
| Labrasol | | 49.49% | | | | |
| | | (65 mg) | | | | |
| Water | | 7.5% | | | | |
| | | (40.1 mg) | | | | |
| Initial | ibuprofen | Clear solution with | Clear solution, | Clear solution | high viscous | ibuprofen |
| Obser- | crystallized | a bubble layer on | yellow color was | with a small piece | clear solution | crystallized |
| vation | and | the top of solution | observed around | of undissolved KOH | | and |
| | precipitated | | the KOH pellet | pellet, yellow color | | precipitated |
| | | | during mixing | was observed | | |
| | | | | as Sample #3 | | |
| Comment | | Additional ibuprofen | Additional ibuprofen | Additional ibuprofen | Additional ibuprofen | KHCO₃ did not work |
| | | added to bring up to | added to bring up to | added to bring up to | added to bring up to | as well as KOH with |
| | | 40%, no crystallization | 40%, no crystallization | 40%, no crystallization | 40%, precipitation | ibuprofen in the |
| | | was observed after two | was observed after two | was observed after two | occurred/ | corn oil based |
| | | days. Further addition | days. Further addition | days. Further addition | | vehicle |
| | | of API to increase | of API to increase | of API to increase | | |
| | | concentration to 50%, | concentration to 50%, | concentration to 50%, | | |
| | | precipitation occurred. | precipitation occurred. | few crystals formed | | |
| | | | | and precipitated. | | |

### Example 5

### Screening for Ibuprofen Solubility in Different Vehicles

Different solvent systems such as oils and medium chain triglycerides, solubilizing agents, emulsifying agents (self-emulsifying) and surfactants for the liquid filled capsules were screened for their effectiveness in solubilizing ibuprofen and are listed in Tables 5 to 6.

Initially, 250.0 mg of ibuprofen was added into 10 g of vehicle individually at room temperature and mixed until a fixed quantity of the drug is solubilized or a suspension was obtained. The screening method involved the following steps:

1. Step T1: on obtaining a clear solution, additional incremental quantities of ibuprofen were added and mixed until a clear solution was obtained.

2. Step T2: the sample was heated using a hot plate and stirrer to facilitate ibuprofen solubility in the vehicle and the samples were left overnight.

3. Step T3: additional ibuprofen was added into the clear solution from step T2 while heating and stirring. Visual observations were made to determine if the mixture could remain as clear solution or crystallized within an approximately 24 hour period. Additional ibuprofen was added by reheating selected T2 solutions until re-crystallization or precipitation was observed.

Observations on whether ibuprofen appeared as dissolved, a suspension (fine suspension) or crystallized (agglomerates) were reported.

TABLE 5
Single Component Vehicle Screening
for Ibuprofen Solubility Study

| | Excipient | Amt of API | |
|---|---|---|---|
| Item No. | (10.0 g) | added (mg) | Observation |
| 1 | Corn Oil | 250.0 | Dissolved (T1) |
| | | 500.0 | Dissolved (T2) |
| | | 2500.0 | Crystallized (T3) |
| 2 | Olive Oil | 250.0 | Dissolved (T1) |
| | | 500.0 | Suspension (T2) |
| | | 2500.0 | Crystallized (T3) |
| 3 | Castor Oil | 257.8 | Solidify (T1) |
| | | N/A | N/A |
| | | N/A | N/A |
| 4 | Akomed R | Up to 500.0 | Dissolved (T1) |
| | | 750.0 | Crystallized (T2) |
| | | N/A | N/A |
| 5 | Captex 355 | Up to 500.0 | Dissolved (T1) |
| | | 1000.0 | Crystallized (T2) |
| | | N/A | N/A |
| 6 | Captex 200P | 500.0 | Dissolved (T1) |
| | | 1000.0 | Dissolved (T2) |
| | | 3000.0 | Crystallized (T3) |
| 7 | Labrafac CC | 500.0 | Dissolved (T1) |
| | | 1000.0 | Crystallized (T2) |
| | | N/A | N/A |
| 8 | Labrafac PG | 500.0 | Dissolved (T1) |
| | | 1000.0 | Dissolved (T2) |
| | | 3000.0 | Crystallized (T3) |
| 9 | Labrasol | 1000.0 | Dissolved (T1) |
| | | 2000.0 | Crystallized (T2) |
| | | N/A | N/A |
| 10 | Miglyol 812 | 500.0 | Dissolved (T1) |
| | | 1000.0 | Dissolved (T2) |
| | | 3250.0 | Crystallized (T3) |
| 11 | Miglyol 840 | 500.0 | Dissolved (T1) |
| | | 750.0 | Dissolved (T2) |
| | | 3000.0 | Crystallized (T3) |
| 12 | Capryol 90 | 1000.0 | Dissolved (T1) |
| | | 2000.0 | Crystallized (T2) |
| | | N/A | N/A |
| 13 | Cremophor RH 40 | 2250.0 | Dissolved (T1) |
| | | 3250.0 | Dissolved (T2) |
| | | 4975.0 | Dissolved (T3) |
| 14 | Imwitor 491 | 250.0 | Solidify (T1) |
| | | N/A | N/A |
| | | N/A | N/A |
| 15 | Imwitor 988 | 250.0 | Suspension (T1) |
| | | 250.0 | Dissolved (T2) |
| | | 3750.0 | Crystallized |
| 16 | Labrafil M1944 CS | 500.0 | Dissolved (T1) |
| | | 1000.0 | Dissolved (T2) |
| | | 3000.0 | Crystallized (T3) |
| 17 | Labrafil M 2125 CS | 500.0 | Dissolved (T1) |
| | | 1000.0 | Dissolved (T2) |
| | | 3000.0 | Crystallized (T3) |
| 18 | Capmul PG8 | 1000.0 | Dissolved (T1) |
| | | 2000.0 | Crystallized (T2) |
| | | N/A | N/A |
| 19 | Plurol Oleique CC 495 | 250.0 | Dissolved (T1) |
| | | 750.0 | Dissolved (T2) |
| | | 3500.0 | Crystallized (T3) |
| 20 | Poloxamer 124 | 250.0 | Dissolved (T1) |
| | | 500.0 | Dissolved (T2) |
| | | 3250.0 | Dissolved (T3) |
| 21 | Softigen 701 | 500.0 | Dissolved (T1) |
| | | 1000.0 | Dissolved (T2) |
| | | 3750.0 | Crystallized (T3) |
| 22 | Solutol HS 15 | 2500.0 | Dissolved (T1) |
| | | 4500.0 | Dissolved (T2) |
| | | 7750.0 | Crystallized (T3) |
| 23 | Tween 80 | 1750.0 | Dissolved (T1) |
| | | 2750.0 | Dissolved (T2) |
| | | 3750.0 | Crystallized (T3) |
| 24 | Tween 20 | 2000.0 | Dissolved (T1) |
| | | 2750.0 | Dissolved (T2) |
| | | 3750.0 | Crystallized (T3) |
| 25 | PEG 400 | 2000.0 | Dissolved (T1) |
| | | 4000.0 | Dissolved (T2) |
| | | 6000.0 | Crystallized (T3) |
| 26 | Propylene Glycol | 1000.0 | Dissolved (T1) |
| | | 2000.0 | Crystallized (T2) |
| | | 4000.0 | Crystallized (T3) |
| 27 | Glycerin | 250.0 | Turbid (T1) |
| | | N/A | N/A |
| | | N/A | N/A |
| 30 | Hexylene Glycol | 4250.0 | Dissolved (T1) |
| | | 5250.0 | Dissolved (T2) |
| | | 10000.0 | Crystallized (T3) |
| 31 | Surfactol 365 | 250.0 | Suspension (T1) |
| | | 250.0 | Dissolved (T2) |
| | | 3500.0 | Crystallized (T3) |
| 33 | Macol LA4 | 2750.0 | Dissolved (T1) |
| | | 4750.0 | Crystallized (T2) |
| | | N/A | N/A |
| 34 | Neobec MS | 750.0 | Dissolved (T1) |
| | | 1500.0 | Crystallized (T2) |
| | | N/A | N/A |
| 35 | Akomed E | 250.0 | Dissolved (T1) |
| | | 1000.0 | Crystallized (T2) |
| | | N/A | N/A |
| 36 | Schercemol TN | 250.0 | Dissolved (T1) |
| | | 1000.0 | Dissolved (T2) |
| | | 3000.0 | Crystallized (T3) |
| 37 | Schercemol DIA | 2250.0 | Dissolved (T1) |
| | | 4250.0 | Crystallized (T2) |
| | | N/A | N/A |
| 38 | Span 80 | 250.0 | Suspension (T1) |
| | | 250.0 | Dissolved (T2) |
| | | 3500.0 | Crystallized (T3) |
| 39 | Myvacel 9-45V | 750.0 | Dissolved (T1) |
| | | 1500.0 | Dissolved (T2) |
| | | 3000.0 | Crystallized (T3) |

TABLE 6
Mixture Vehicles Screening for Ibuprofen Solubility Study

| | Excipient | Amt of API | |
|---|---|---|---|
| Item No. | (10.0 g) | added (mg) | Observation |
| 28 | Phasol 50 PG (5.0 g) + | N/A | N/A |
| | Labrasol (5.0 g) | 6700.0 | Crystallized (T2) |
| | | N/A | N/A |
| 29 | Phosal 53 NCT (5.0 g) + | N/A | N/A |
| | Labrasol (5.0 g) | 6700.0 | Dissolved (T2) |
| | | 6700.0 | Crystallized (T3) |
| 32 | Labrafac Hydro WL 1219 | 1000.0 | Dissolved (T1) |
| | | 4250.0 | Crystallized (T2) |
| | | N/A | N/A |
| 40 | Phosal 50 PG (5.0 g) + | 4500.0 | Dissolved (T1) |
| | Propylene Glycol(5.0 g) | 6500.0 | Crystallized (T1) |
| | | N/A | N/A |
| 41 | Phosal 53 MCT (5.0 g) + | 4500.0 | Dissolved (T1) |
| | Propylene Glycol (5.0 g) | 6500.0 | Crystallized (T2) |
| | | N/A | N/A |
| 42 | Tween 20 (2.0 g) + | 4000.0 | Crystallized (T1) |
| | Phosal 53 MCT (4.0 g) + | N/A | N/A |
| | Ibuprofen (4.0 g) | N/A | N/A |
| 43 | Phosal 53 MCT (5.0 g) + | 5000.0 | Crystallized (T1) |
| | PEG 400 (5.0 g) | N/A | N/A |
| | | N/A | N/A |
| 44 | Phosal 50 PG (5.0 g) + | 5000.0 | Crystallized (T1) |
| | PEG 400 (5.0 g) | N/A | N/A |
| | | N/A | N/A |
| 45 | Phosal 50 PG (5.0 g) + | 5000.0 | Crystallized (T1) |
| | Capmul PG8 (5.0 g) | N/A | N/A |
| | | N/A | N/A |
| 46 | Phosal 53 MCT(5.0 g) + | 5000.0 | Crystallized (T1) |
| | Capmul PG8 (5.0 g) | N/A | N/A |
| | | N/A | N/A |

### Example 6

### Solubility of Ibuprofen in Single Component Vehicles

Liquid vehicles falling in classification of solubilizers, surfactants, fillers, and emulsifiers were chosen as solubilizers to conduct this study. The aim of the study was maximize the solubility of ibuprofen in individual vehicles and combination of solvent systems.

Initially, 250.0 mg of ibuprofen was added into 10 g of vehicle individually at room temperature and mixed until a fixed quantity of the drug is solubilized or a suspension was obtained. The screening method involved the following steps:
1. Step T1: on obtaining a clear solution, additional incremental quantities of ibuprofen were added and mixed until a clear solution was obtained.
2. Step T2: the sample was heated using a hot plate and stirrer to facilitate ibuprofen solubility in the vehicle and the samples were left overnight.
3. Step T3: additional ibuprofen was added into the clear solution from step T2 while heating and stirring. Visual observations were made to determine if the mixture could remain as clear solution or crystallized within an approximately 24 hour period. Additional ibuprofen was added by reheating selected T2 solutions until re-crystallization or precipitation was observed.

Observations on whether ibuprofen appeared as dissolved, a suspension (fine suspension) or crystallised (agglomerates) were reported.

A. Solubility of Ibuprofen in Lecithin (Phosphotidylcholine) Based Mixture Vehicles

Single component vehicles exhibiting satisfactory ibuprofen solubilities were combined with Phosphatidylcholine, Phosal 53 MCT and Phosal 50 PG for further solubility screening. The compositions of the mixture vehicles are presented in Table 7.

TABLE 7
Compositions of Ibuprofen in Phosphatidylcholine Based Mixed Vehicles

| | Compositions of samples (% w/w) | | | | | | | | |
|---|---|---|---|---|---|---|---|---|---|
| | 1 | 2 | 3 | 4 | 5 | 6 | 7 | 8 | 9 |
| | Exp1 | Exp1 | Exp1 | Exp1 | Exp1 | Exp1 | Exp1 | Exp1 | Exp1 |
| Excipients | - 54 | - 55 | - 52 | - 50 | - 57 | - 56 | - 53 | - 38 | - 37 |
| Ibuprofen | 33.3 | 33.3 | 31.1 | 31.0 | 33.3 | 33.3 | 39.8 | 40.1 | 40.1 |
| Phosal 53 MCT | 33.3 | | 34.4 | | 33.3 | | 40.0 | 29.9 | |
| Phosal 50 PG | | 33.3 | | 34.5 | | 33.3 | | | 29.9 |
| PEG 400 | 33.3 | 33.3 | | | | | | | |
| Propylene Glycol | | | 34.4 | 34.5 | | | | | |
| Capmul PG 8 | | | | | 33.3 | 33.3 | | | |
| Tween 20 | | | | | | | 20.0 | | |
| Labrasol | | | | | | | | 29.9 | 29.9 |

Approximately 5 g of ibuprofen was added into 10 g of vehicle mixture in a 20 ml scintillation vial. The mixture was placed on a hot plate with continuous stirring, using a magnetic stiffer, to dissolve the ibuprofen to form a clear solution. Visual observations were made to determine if the ibuprofen would precipitate out in approximately 24 hours. If ibuprofen remained in solution, an incremental quantity of the drug substance was added to the test mixture. The mixture was re-heated to form a solution. Visual observations were repeated until the presence of solid ibuprofen was observed.

B. Solubility of Ibuprofen in Oil/Alkalizing Agent Based Mixture Vehicles

The oils selected for this screening study were polyunsaturated oils including corn oil, soybean oil, sunflower oil, sesame oil, peanut oil, cottonseed oil, olive oil and peppermint oil. Alkalizing agents such as Potassium hydroxide and potassium bicarbonate were used to enhance the solubility of Ibuprofen in oils by reacting with Ibuprofen to form a salt. The compositions of the mixed vehicles arc presented in Table 8.

The samples were prepared by adding approximately 4 g of ibuprofen into 5.65 g of oil in a scintillation vial. The obtained viscous liquid was heated using a hot plate. Ibuprofen started dissolving in the vehicle and a clear solution was obtained. To the clear solution approximately 0.33 g of Potassium hydroxide pellets (KOH) or potassium bicarbonate was added slowly with continuous mixing and heating. After the KOH melted completely, the samples were stirred for another 10 minutes and then allowed to cool down to room temperature. On a regular basis visual observations were carried out to check for the presence of solid ibuprofen. Additional ibuprofen was added to the solution while mixing and heating continuously to check for the maximum amount of ibuprofen dissolved at room temperature.

C. Ibuprofen in solvent/Solubilizer/Alkalizing Agent Based Mixture Vehicles

Ibuprofen was also studied in different solvent systems with alkalizing agents such as potassium hydroxide or potassium bicarbonate.

TABLE 8
Compositions of Ibuprofen in Oil/Alkalizing Agent Based Mixture Vehicles

| | Compositions of samples (% w/w) | | | | | | | | | | |
|---|---|---|---|---|---|---|---|---|---|---|---|
| | 1 | 2 | 3 | 4 | 5 | 6 | 7 | 8 | 9 | 10 | 11 |
| | Exp 2 | Exp 1-1 | Exp 2 | Exp 2 | Exp 2 | Exp 2 | Exp 2 | Exp 2 | Exp 2 | Exp 2 | Exp 3 |
| Ingredient | #1 | #3A | #2 | #8 | #9 | #10 | #11 | #12 | #16 | #18 | #12 |
| Ibuprofen | 40.0 | 35.0 | 40.0 | 40.0 | 40.0 | 40.0 | 40.0 | 40.0 | 49.7 | 43.2 | 50.3 |
| Corn oil | 56.5 | 60.0 | | | | | | | 41.0 | 43.4 | |
| Soybean oil | | | 56.5 | | | | | | | | 41.1 |
| Sunflower oil | | | | 56.5 | | | | | | | |
| Sesame oil | | | | | 56.5 | | | | | | |
| Peanut oil | | | | | | 56.5 | | | | | |
| Cottonseed oil | | | | | | | 56.5 | | | | |
| Olive oil | | | | | | | | 56.5 | | | |
| Peppermint oil | | | | | | | | | 4.7 | | |
| Ethanol | | | | | | | | | | 9.8 | 4.5 |
| Potassium | | 5.4 | | | | | | | | | |
| bicarbonate | | | | | | | | | | | |
| Potassium | 3.5 | | 3.5 | 3.5 | 3.5 | 3.5 | 3.5 | 3.5 | 4.6 | 3.6 | 4.1 |
| hydroxide | | | | | | | | | | | |

The samples were prepared in clear glass scintillation vials. Vehicle systems were designed from previous solubility studies. Ibuprofen was added to the vehicle with continuous heating and mixing. Potassium hydroxide (or potassium bicarbonate) was added to the liquid with continuous mixing and heating and the samples were checked visually for solubility of ibuprofen. Samples were kept for overnight to check the crystallization of ibuprofen from the vehicle system. Visual observation carried out for possible crystallization, which thus indicated that the saturation point of vehicle system had been reached. The formulation compositions of the samples are provided in Table 9.

TABLE 9
Formulation Compositions of Ibuprofen in Solvent/Solubilizer/Alkalizing Agent Vehicles

| | Compositions of samples (% w/w) | | | | | | | | | | | |
|---|---|---|---|---|---|---|---|---|---|---|---|---|
| | | | | 4 | | | | | | | | |
| | 1 | | 3 | Exp2 | | | | | | | | |
| | Exp1 | 2 | Exp1 | #13 | 5 | 6 | 7 | 8 | 9 | 10 | 11 | 12 |
| | #1 | Exp2 | #1 | (U.S. Pat. | Exp2 | Exp2 | Exp2 | Exp2 | Exp3 | Ex3 | Exp3 | Exp3 |
| Ingredient | (EP1344523) | #17 | (WO02069936) | No. 5360615) | #7 | #23 | #25 | #26 | #8 | #9 | #7 | #10 |
| Ibuprofen | 47.6 | 45.4 | 37.34 | 67.0 | 50 | 49.8 | 45.5 | 49.3 | 50.0 | 50.1 | 50.2 | 50.1 |
| PEG400 | | | | 16.7 | | | | | | | | |
| Propylene | | | | 3.3 | | | | | | | | |
| glycol | | | | | | | | | | | | |
| Soybean oil | | | | | | | | | | | 22.6 | 35.0 |
| Solutol HS 15 | | | | | 46 | 36.9 | 40.6 | | | | | |
| Cremophor | 33.5 | 31.9 | | | | | | | | | | |
| RH40 | | | | | | | | | | | | |
| Miglyol 812 | 15.0 | 14.6 | | | | | | | 15.0 | | | |
| Labrasol | | | 49.5 | | | | | 28.3 | | | | |
| Gelucire 44/14 | | | | | | | | 18.3 | 30.2 | 35.0 | 22.5 | |
| Tween 80 | | | | | | | | | | 10.0 | | 10.1 |
| Phosal 50 PG | | | | | | 9.3 | | | | | | |
| Phospholipon | | | | | | | 10.2 | | | | | |
| 90 G | | | | | | | | | | | | |
| KOH | 3.9 | 3.7 | | 5.6 | 4.0 | 4.0 | 3.7 | 4.1 | 4.8 | 4.8 | 4.7 | 4.8 |
| KHCO3 | | | 5.43 | | | | | | | | | |
| PVP 29/31 | | | | 5.0 | | | | | | | | |
| Water | | 4.4 | 7.5 | 6.4 | | | | | | | | |

### Example 7

### Prototype Batches

A. Vehicle Selection and Evaluation

Five Prototype batches were manufactured to further study the solubility of ibuprofen. The total batch size was 50 g. The vehicle was heated using a hot plate up to 80° C. with continuous mixing. Ibuprofen was added by slow addition and mixed for approximately 20 minutes at 60 to 80° C. This solution was stored at room temperature and visually observed. Ibuprofen solutions were encapsulated in size 00 Gelatin and HPMC capsules using a manual encapsulator MF 30 and an Eppendorf micropipette. Gelatin capsules were filled to a target weight of 915 mg and HPMC capsules were filled to a target weight of 930 mg and tested for dissolution and ibuprofen content. The compositions of the prototype batches are detailed in Table 10.

TABLE 10
Prototype Batches for Solubility Evaluation

| Lot Number | Formulation | % w/w |
|---|---|---|
| MCNLF4000101 | ibuprofen | 50.0 |
| | Solutol HS 15 | 45.2 |
| | KOH | 4.8 |
| MCNLF4000301 | Ibuprofen | 50.0 |
| | Labrasol | 27.2 |
| | Gelucire 44/14 | 18.0 |
| | KOH | 4.8 |
| MCNLF4000401 | Ibuprofen | 50.0 |
| | Phosal 50 PG | 10.0 |
| | Solutol HS 15 | 35.2 |
| | KOH | 4.8 |
| MCNLF4000501 | Ibuprofen | 50.0 |
| | Phospholipon 90G | 9.0 |
| | SolutolHS 15 | 36.2 |
| | KOH | 4.8 |
| MCNLF4000601 | Ibuprofen | 45.4 |
| | Cremophor RH 40 | 31.9 |
| | Miglyol 812 | 14.6 |
| | KOH | 3.7 |
| | Water | 4.4 |

B. Freeze Thaw Study for Prototype Batches

Based on the initial vehicle screening study, the five solvent systems (Table 10) were selected and prepared for a freeze-thaw study.

Two samples, 10 g for each formulation, packaged in 20 ml scintillation glass vials and closed with a plastic cap were refrigerated at 2° C. to 8° C. for 48 hours followed by storing at ambient room temperature for 48 hours for three cycles. The samples were examined visually and microscopically for possible Ibuprofen solids and change in color after each cycle. Table 11 lists the storage condition and test time point for three cycles.

TABLE 11
Storage Conditions and Test Time Points for
Freeze/Thaw Study on Prototype Batches

| Time Point | Storage Times and Conditions | No. of Samples |
|---|---|---|
| T = 0 | N/A | 2 |
| Cycle 1 | 48 hrs at 5 ± 3° C./Ambient RH, | 2 |
| Cycle 2 | 48 hrs at 5 ± 3° C./Ambient RH, | 2 |
| Cycle 3 | 48 hrs at 5 ± 3° C./Ambient RH | 2 |

N/A = Not Applicable

No precipitation or crystallisation of ibuprofen for prototype batches MCNLF4000101, MCNLF4000301, MCNLF4000401, MCNLF4000501 and MCNLF4000601 was observed visually and by optical microscope during and after the three freeze-thaw cycles.

C. Dissolution Study

The prototype mixtures were filled into five formulations, which were filled into gelatin and HPMC capsules (size 00) to check the maximum fill quantities. Approximately 950 mg of ibuprofen solution was filled for all five prototype formulations and analyzed for ibuprofen content and in-vitro dissolution according to the dissolution method outlined in United States Pharmacopeia (USP 23) for ibuprofen tablets.

The ibuprofen content was satisfactory for prototype mixtures (Table 12). The ibuprofen content (mg per capsule) and % release of ibuprofen in-vitro dissolution results after 60 minutes are provided in Table 13.

TABLE 12
Prototype Mixture Potency Results

| | | % LC of | | |
|---|---|---|---|---|
| | | Ibuprofen | Mean | Results |
| Lot # | Sample # | Released | % LC | % w/w |
| MCNLF4000101 | 1 | 100.5 | 100.5 | 50.3 |
| | 2 | 100.4 | | |
| MCNLF4000301 | 1 | 100.4 | 100.4 | 50.2 |
| | 2 | 100.3 | | |
| MCNLF4000401 | 1 | 100.2 | 100.4 | 50.2 |
| | 2 | 100.6 | | |
| MCNLF4000501 | 1 | 100.7 | 100.7 | 50.4 |
| | 2 | 100.6 | | |
| MCNLF4000601 | 1 | 102.4 | 102.6 | 46.6 |
| | 2 | 102.8 | | |

LC = Label Claim

TABLE 13
Ibuprofen Content and In-Vitro Dissolution Results for Encapsulated Ibuprofen Capsules

| Capsule | | | Ibuprofen | % Release after | Observation at |
|---|---|---|---|---|---|
| Type | Lot # | Capsule # | mg/capsule | 60 minutes | 60 minutes |
| Gelatin | MCNLF4000101 | 1 | 468.6 | 97.9 | Clear, small shell piece |
| | | | | | seen |
| | | 2 | 467.6 | 103.4 | Clear, dissolved completely |
| | MCNLF4000301 | 1 | 458.6 | 105.8 | Little hazy, small shell |
| | | | | | piece seen |
| | | 2 | 468.0 | 131.4 | Little hazy, dissolved |
| | | | | | completely |
| | MCNLF4000401 | 1 | 455.2 | 106.2 | Hazy, white particles and |
| | | 2 | 440.9 | 102.9 | small shell piece seen for |
| | | | | | both capsules |
| | MCNLF4000501 | 1 | 466.9 | 102.5 | Very hazy, small shell |
| | | 2 | 464.7 | 111.1 | piece seen for both |
| | | | | | capsules |
| | MCNLF4000601 | 1 | 441.2 | 102.0 | Clear, initially white |
| | | 2 | 456.2 | 102.6 | particles seen which |
| | | | | | dissolved towards the end |
| HPMC | MCNLF4000101 | 1 | 465.0 | 74.1 | Hazy, small shell piece |
| | | 2 | 465.4 | 101.1 | seen for both capsules |
| | MCNLF4000301 | 1 | 458.1 | 26.4 | Hazy, small shell piece |
| | | 2 | 459.0 | 61.2 | seen for both capsules |
| | MCNLF4000401 | 1 | 462.5 | 105.7 | Hazy, small shell piece |
| | | 2 | 468.8 | 103.1 | seen for both capsules |
| | MCNLF4000501 | 1 | 460.9 | 59.1 | Clear, capsule seems |
| | | 2 | 469.7 | 35.1 | swollen and not deformed |
| | | | | | till end |
| | MCNLF4000601 | 1 | 454.6 | 12.2 | Clear, capsule seems |
| | | 2 | 453.1 | 12.5 | swollen and not deformed |
| | | | | | till end |

D. Maximum Solubility Study

Based on the earlier vehicle screening, solubility and freeze thaw studies on previous prototype batches that incorporated approximately 50% w/w ibuprofen, (six formulations) were selected to further maximize the solubility of ibuprofen in selected vehicles, such that the capsule size could be reduced from a size 00 to 1. Different concentrations of ibuprofen, 50% w/w, 55% w/w, 60% w/w, 65% w/w and 68% w/w, in different vehicle systems (15-20 g) were mixed while heating at 58° C.±5° C. in 20 ml scintillation vials. Potassium hydroxide pellets were dissolved by mixing with continuous heating for 40 minutes at 75° C.±5° C. The solution was cooled to ambient temperature and the solubility of ibuprofen was visually confirmed. These solutions were divided into two equal parts and stored at ambient room temperature and at refrigerated conditions at t=0, 24 hours and 3, 4 and 6 days and observed for precipitation/recrystallisation. The compositions are provided in the Table 14.

TABLE 14
Prototype Formulations Containing 50%, 55%, 60%, 65% and 68% w/w Ibuprofen

| Formulation | | | | | |
|---|---|---|---|---|---|
| Composition | MCNLF4000701 | MCNLF4000702 | MCNLF4000703 | MCNLF4000704 | MCNLF4000705 |
| Ibuprofen | 49.9 | 55.0 | 60.0 | 64.9 | 68.1 |
| Solutol HS 15 | 45.9 | 41.3 | 36.7 | 32.2 | 26.3 |
| Potassium | 4.2 | 3.7 | 3.3 | 2.9 | 5.6 |
| Hydroxide | | | | | |

| | MCNLF4000801 | MCNLF4000802 | MCNLF4000803 |
|---|---|---|---|
| Ibuprofen | 50.0 | 54.9 | 59.9 |
| Labrasol | 28.0 | 25.2 | 22.3 |
| Gelucire 44/14 | 18.0 | 16.3 | 14.5 |
| Potassium | 4.0 | 3.6 | 3.3 |
| Hydroxide | | | |

| | MCNLF4000901 | MCNLF4000902 | MCNLF4000903 |
|---|---|---|---|
| Ibuprofen | 49.9 | 55.0 | 60.0 |
| Phospholipon 90G | 9.2 | 8.3 | 7.6 |
| Solutol HS 15 | 37.0 | 33.3 | 29.4 |
| Potassium | 3.9 | 3.4 | 3.0 |
| Hydroxide | | | |

| | MCNLF4001001 | MCNLF4001002 | MCNLF4001003 | MCNLF4001004 |
|---|---|---|---|---|
| Ibuprofen | 49.8 | 54.9 | 60.0 | 65.0 |
| Phosal 50 PG | 9.3 | 8.4 | 7.5 | 6.5 |
| Solutol HS 15 | 36.8 | 33.1 | 29.3 | 25.7 |
| Potassium | 4.1 | 3.6 | 3.2 | 2.8 |
| Hydroxide | | | | |

| | MCNLF4001101 | MCNLF4001102 |
|---|---|---|
| Ibuprofen | 50.0 | 55.0 |
| Corn Oil | 46.0 | 41.4 |
| Potassium | 4.0 | 3.6 |
| Hydroxide | | |

| | MCNLF4001201 | MCNLF4001202 |
|---|---|---|
| Ibuprofen | 49.9 | 55.0 |
| Cremophore RH 40 | 29.2 | 26.3 |
| Miglyol 812 | 13.4 | 12.0 |
| Potassium | 3.4 | 3.0 |
| Hydroxide | | |
| Water | 4.1 | 3.7 |

Six Prototype batches (MCNLF4000701, MCNLF4000801, MCNLF4000901, MCNLF4001001, MCNLF4001101 and MCNLF4001201) containing approximately 50% w/w ibuprofen exhibited clear solutions when stored at room temperature at t=0 and t=24 hours and in the refrigerator for 24 hours. Ibuprofen solids were observed for prototype batches MCNLF4001101 and MCNLF4001201 after storage at room temperature and at refrigerated conditions for three months.

The t=0 samples were clear at room temperature for all six prototype batches (MCNLF4000702, MCNLF4000802, MCNLF4000902, MCNLF4001002, MCNLF4001102 and MCNLF4001202). Prototype batches MCNLF4000702 to MCNLF4001002 exhibited clear solutions after 24 hours at room temperature and refrigerated conditions. However, Prototype batches MCNLF4001102 and MCNLF4001202 exhibited crystallization after storage for 24 hours at room temperature as well as refrigerated conditions. Since Prototype batches MCNLF4001102 and MCNLF4001202 exhibited crystallization at 55% w/w ibuprofen content, these two prototype formulations were not considered for further evaluation of maximum solubility. Prototype batch MCNLF4000702 remained clear after three months storage at room temperature and refrigerated conditions.

Four Prototype batches, MCNLF4000703, MCNLF4000803, MCNLF4000903 and MCNLF4001003, containing approximately 60% w/w of ibuprofen remained visually clear when stored at room temperature at t=0. Precipitates were observed for prototype batch MCNLF4000903 after 24 hours of storage at room temperature. Prototype batches MCNLF4000803 and MCNLF4000903 produced crystals after 24 hours of storage at refrigerated conditions, therefore, these prototypes were not considered for further evaluation. Prototype batches MCNLF4000703, MCNLF4000803 and MCNLF4001003 were clear solutions after three months storage at room temperature.

Two prototype batches, MCNLF4000704 and MCNLF4001004, contained approximately 65% w/w ibuprofen. Prototype batch MCNLF4000704, containing ibuprofen (65% w/w), Solutol Hs 15 (32.1% w/w) and Potassium hydroxide (2.9% w/w) was visually clear only at the t=0 room temperature condition. Crystallization occurred at the room temperature and refrigerated storage conditions. Prototype batch MCNLF4001004, containing ibuprofen (65% w/w), Phosal 50 PG (6.5% w/w), Solutol HS 15 (25.7% w/w) and Potassium hydroxide (2.8% w/w) exhibited crystallization at all storage conditions.

Both prototype batches exhibited crystallization after three months storage at room temperature and refrigerated conditions.

One prototype batch MCNLF000705 containing approximately 68% w/w ibuprofen was evaluated and it exhibited crystallization at all storage conditions.

The formulation composition containing Solutol HS15 was further evaluated for equilibrium solubility studies (Prototype series 7).

### Example 8

### Equilibrium Solubility Studies

Based on the vehicle screening, solubility study, freeze thaw study and a maximum solubility study, a formulation containing ibuprofen, Solutol HS15 and potassium hydroxide pellets was chosen for further equilibrium solubility studies. The aim of the study was to evaluate the equilibrium solubility of ibuprofen in the given vehicle system by varying the concentration of Solutol HS15, Potassium hydroxide and Water. In this study ibuprofen concentration was kept constant at 60% w/w. Three levels of potassium hydroxide (4.1% w/w, 5.3% w/w and 6.5% w/w), Water (0.0% w/w, 1.5% w/w and 3.0% w/w) and Solutol HS 15 (34.7% w/w, 33.5% w/w and 31.7% w/w) were chosen. Thirteen batches were prepared at a batch size of 15 g (MCNLF4000706 to MCNLF4000718). The formulation compositions are provided in Table 12.

Ibuprofen and Solutol HS 15 were mixed in 20 ml scintillation vials while heating at 85° C.±5° C. Potassium Hydroxide pellets were added with continuous mixing and heating for 30 minutes at 75° C.±5° C. Purified water was added as required with continuous mixing and heating. The temperature of the solution was allowed to cool at ambient temperature and the samples were stored at 2-8° C. in the refrigerator for seven days. The same samples were removed from the refrigerator and stored at ambient room temperature for another 5 days (total 12 days). Visual observations were carried out to confirm the solubility of ibuprofen at the initial time-point, 24 hours, 5, 7, 9 and 12 days (Table 17). The samples were further subjected to a freeze-thaw study to evaluate possible precipitation/recrystallisation of ibuprofen at room temperature. The formulation compositions for this study are provided in the Table 15.

TABLE 15
Formulation Compositions for Equilibrium Study

| | | Formulation Compositions (% w/w) | | | |
|---|---|---|---|---|---|
| | | | Solutol | Potassium | |
| # | Batch Number | Ibuprofen | HS 15 | Hydroxide | Water |
| 1 | MCNLF4000706 | 60.0 | 31.7 | 5.3 | 3.0 |
| 2 | MCNLF4000707 | 60.0 | 34.7 | 5.3 | 0.0 |
| 3 | MCNLF4000708 | 60.0 | 32.0 | 6.5 | 1.5 |
| 4 | MCNLF4000709 | 60.0 | 30.5 | 6.5 | 3.0 |
| 5 | MCNLF4000710 | 60.0 | 33.2 | 5.3 | 1.5 |
| 6 | MCNLF4000711 | 60.0 | 35.9 | 4.1 | 0.0 |
| 7 | MCNLF4000712 | 60.0 | 33.5 | 6.5 | 0.0 |
| 8 | MCNLF4000713 | 60.0 | 34.4 | 4.1 | 1.5 |
| 9 | MCNLF4000714 | 60.0 | 32.9 | 4.1 | 3.0 |
| 10 | MCNLF4000715 | 60.0 | 33.2 | 5.3 | 1.5 |
| 11 | MCNLF4000716 | 60.0 | 33.3 | 4.9 | 1.8 |
| 12 | MCNLF4000717 | 60.0 | 32.9 | 4.1 | 3.0 |
| 13 | MCNLF4000718 | 60.0 | 32.9 | 4.1 | 3.0 |

The analytical initial solubility results are provided in Table 16. Visual observations were also performed at different time intervals of 24 hours, 5 and 7 days at 2-8° C., 9 and 12 days at ambient room temperature (Table 17). Prototype batches containing ibuprofen, Solution HS 15 and Potassium hydroxide, MCNLF4000708, MCNLF4000709, MCNLF4000710 and MCNLF4000715, remained as clear solutions throughout the 12 days storage period. This could be due to the presence of equimolar concentrations of potassium hydroxide in relation to the ibuprofen concentration. To attain a maximum solubility for ibuprofen in the solvent system, Potassium hydroxide in an equimolar concentration, is necessary to complete the reaction between ibuprofen and potassium hydroxide for solubilisation in Solutol HS15.

TABLE 16
Analytical Initial Ibuprofen Content, mg/ml

| | Ibuprofen Content, | |
|---|---|---|
| Lot # | (mg/ml) | % w/w |
| MCNLF4000706 | 650.2 | 60.3 |
| MCNLF4000707 | 632.0 | 60.5 |
| MCNLF4000708 | 672.3 | 61.0 |
| MCNLF4000709 | 645.2 | 61.0 |
| MCNLF4000710 | 659.2 | 60.9 |
| MCNLF4000711 | 576.1 | 59.1 |
| MCNLF4000712 | 646.6 | 60.5 |
| MCNLF4000713 | 661.9 | 60.4 |
| MCNLF4000714 | 583.3 | 53.5 |
| MCNLF4000715 | 645.1 | 60.7 |
| MCNLF4000716 | 644.7 | 60.7 |
| MCNLF4000717 | 643.9 | 61.2 |
| MCNLF4000718 | 652.2 | 60.9 |

TABLE 17
Visual Observations of Ibuprofen Formulations

| | Clear at | Precipitation | Precipitation | Precipitation |
|---|---|---|---|---|
| Lot # | 12 days | at 5 days | 7 days | 12 days |
| MCNLF4000706 | | ✓ | | |
| MCNLF4000707 | | | | ✓ |
| MCNLF4000708 | ✓ | | | |
| MCNLF4000709 | ✓ | | | |
| MCNLF4000710 | ✓ | | | |
| MCNLF4000711 | ✓ | ✓ | | |
| MCNLF4000712 | | | | ✓ |
| MCNLF4000713 | | ✓ | | |
| MCNLF4000714 | | | ✓ | |
| MCNLF4000715 | ✓ | | | |
| MCNLF4000716 | ✓ | | | ✓ |
| MCNLF4000717 | | ✓ | | |
| MCNLF4000718 | | ✓ | | |

Batches MCNLF4000708, MCNLF4000709, MCNLF4000710 were further tested for water activity, using a Rotronic Hygrolab, as these formulations contained varying amounts of water along with batch MCNLF4000712 as a control (contained no water). The results are provided in Table 18. As seen in the control batch MCNLF4000712, a reaction between potassium hydroxide and bound water in the ibuprofen appears to dissolve the potassium hydroxide.

TABLE 18
Water Activity Results

| | % w/w Purified | | |
|---|---|---|---|
| Correct Batch | Water in the | AW | Temperature |
| Number | Formulation | Reading | (° C.) |
| Silica Gel | | 0.072 | 19.7 |
| Water | | 0.999 | 19.56 |
| MCNLF4000708 | 1.5 | 0.647 | 19.68 |
| MCNLF4000709 | 3.0 | 0.754 | 19.68 |
| MCNLF4000710 | 1.5 | 0.614 | 19.87 |
| MCNLF4000712 | 0.0 | 0.508 | 19.64 |

The scope of the present invention is not limited by the description, examples, and suggested uses herein and modifications can be made without departing from the spirit of the invention. Thus, it is intended that the present invention cover modifications and variations of this invention provided that they come within the scope of the appended claims and their equivalents. Unless otherwise defined, all technical and scientific terms used herein have the same meaning as commonly understood by one of ordinary skill in the art to which this invention pertains. All publications, patent applications, patents, and other references mentioned herein are incorporated reference in their entirety. In case of conflict, the present specification, including definitions, will control.

What is claimed is:

1. A pharmaceutically acceptable solution for filling a hard capsule comprising, based upon the total weight of the solution:
(a) from about 45 to about 75% by weight ibuprofen,
(b) from about 3 to about 5% by weight of an alkylizing agent, and
(c) from about 30 to about 46% by weight of a solvent selected from the group consisting of a vegetable oil, a polyglycolized glyceride, a combination of polyethylene glycol and a polyoxyethylene stearate, and combinations thereof,
wherein the molar ratio between the alkylizing agent and ibuprofen is about 1:about 1.

2. The solution of claim 1, wherein ibuprofen is present in an amount of about 60% by weight.

3. The liquid filled hard capsule of claim 1, wherein the alkylizing agent is an alkali hydroxide and is present in an amount of about 4% by weight.

4. A pharmaceutically acceptable solution for filling a hard capsule comprising, based upon the total weight of the solution:
(a) from about 60% by weight ibuprofen,
(b) from about 3% by weight of potassium hydroxide and
(c) from about 37% by weight of a combination of polyethylene glycol and a polyoxyethylene stearate,
wherein the molar ratio between the potassium hydroxide and ibuprofen is about 1:about 1.
